(19) United States
(12) Patent Application Publication
(10) Pub. No.: US 2022/0079635 A1
(43) Pub. Date: Mar. 17, 2022

(54) LAG-SCREW-RETAINING CLIP AND NAIL SYSTEMS AND METHODS INCORPORATING THE SAME

(71) Applicant: Spinal Generations LLC, Mokena, IL (US)

(72) Inventor: Patrick J. Sweeney, Flossmoor, IL (US)

(73) Assignee: Spinal Generations LLC, Mokena, IL (US)

(21) Appl. No.: 17/444,966

(22) Filed: Aug. 12, 2021

Related U.S. Application Data
(60) Provisional application No. 62/706,448.
(60) Provisional application No. 63/079,011, filed on Sep. 16, 2020.

(51) Int. Cl. A61B 17/74; A61B 17/86

(52) U.S. Cl. CPC A61B 17/744; A61B 17/8695

(57) ABSTRACT

One aspect of the invention provides a lag-screw-retaining clip including: a medial flange adapted and configured for engagement medially under a head of a laterally inserted screw of a trochanteric fracture nail system; an axial offset member coupled to the medial flange, the axial offset member including: a first, medial end coupled to the medial flange; and a second, lateral end; and a lateral flange coupled to the second, lateral end of the axial offset member. The lateral flange is adapted and configured to rest against a lateral end of a lag screw of the trochanteric fracture nail system and resist lateral movement of the lag screw.

### CROSS-REFERENCE TO RELATED APPLICATION

This application claims the benefit of priority under 35 U.S.C. § 119(e) of U.S. Provisional Patent Application Ser. Nos. 62/706,448, filed Aug. 18, 2020, and 63/079,011, filed Sep. 16, 2020. The entire content of each of these applications is hereby incorporated by reference herein.

### BACKGROUND OF THE INVENTION

Proximal femoral fractures are serious injuries that often require surgery to repair. While the type of surgery depends on the location and severity of the fracture, the alignment of the broken bones, and the age and underlying health conditions of the patient, internal nails and screws are frequently used at the site of the fracture.

### SUMMARY OF THE INVENTION

One aspect of the invention provides a lag-screw-retaining clip including: a medial flange adapted and configured for engagement medially under a head of a laterally inserted screw of a trochanteric fracture nail system; an axial offset member coupled to the medial flange, the axial offset member including: a first, medial end coupled to the medial flange; and a second, lateral end; and a lateral flange coupled to the second, lateral end of the axial offset member. The lateral flange is adapted and configured to rest against a lateral end of a lag screw of the trochanteric fracture nail system and resist lateral movement of the lag screw.

This aspect of the invention can have a variety of embodiments. The medial flange and the lateral flange can be positioned at right angles with respect to the axial offset member. The medial flange can be an annular washer. The lateral flange can be an annular washer. The lateral flange can include a medial boss adapted and configured to engage with the lateral end of the lag screw. The lateral flange can include a complementary geometry adapted and configured to engage with the lateral end of the lag screw.

The lag-screw-retaining clip can consist of a biocompatible material. The biocompatible material can be selected from the group consisting of: titanium alloy, stainless steel, and PEEK.

The axial offset member can include a medial bevel. The axial offset member can include a lateral bevel.

Another aspect of the invention provides a trochanteric fracture nail system including: a trochanteric fracture nail; a lag screw adapted and configured for fixation medially through the trochanteric fracture nail; a laterally inserted screw adapted and configured for fixation medially through the trochanteric fracture nail; and the lag-screw-retaining clip as described herein.

This aspect of the invention can have a variety of embodiments. The lag screw and the laterally inserted screw can be adapted for fixation parallel to each other. The trochanteric fracture nail can include two parallel bores.

The trochanteric fracture nail system can further include a guide comprising parallel bores for placement of the lag screw and the laterally inserted screw through a subject's femur and through the trochanteric fracture nail. The guide can be adapted and configured to engage with a superior end of the trochanteric fracture nail.

Another aspect of the invention provides a method of treating a proximal femoral fracture in a subject in need of treatment. The method includes: placing a trochanteric fracture nail axially within the subject's femur; driving a lag screw through the femur and through the trochanteric fracture nail; placing the lateral flange of the lag-screw-retaining clip as described herein laterally over a lateral end of the lag screw such that the medial flange lies medial to the lateral flange; and driving a laterally inserted screw through the femur and through the trochanteric fracture nail such that: the medial flange of the lag-screw-retaining clip is laterally impinged by the laterally inserted screw; and the lateral flange of the lag-screw-retaining clip laterally impinges the lag screw.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIGS. 1A-1D provide several views of a subject's femur comprising a trochanteric fracture nail system according to an embodiment of the invention.

FIGS. 2A-2B provide views of a trochanteric fracture nail system further comprising a guide according to an embodiment of the invention.

FIGS. 3A-3B provide several views of a lag-screw-retaining clip according to an embodiment of the invention.

FIG. 4 depicts a method of treating a proximal femoral fracture in a subject in need thereof in accordance with a preferred embodiment of the invention.

FIG. 5 provides views of a superior end of a subject's femur comprising a trochanteric fracture nail system wherein the views depict the portion of the nail system that protrudes from the femur after the nail system is placed into the subject's femur.

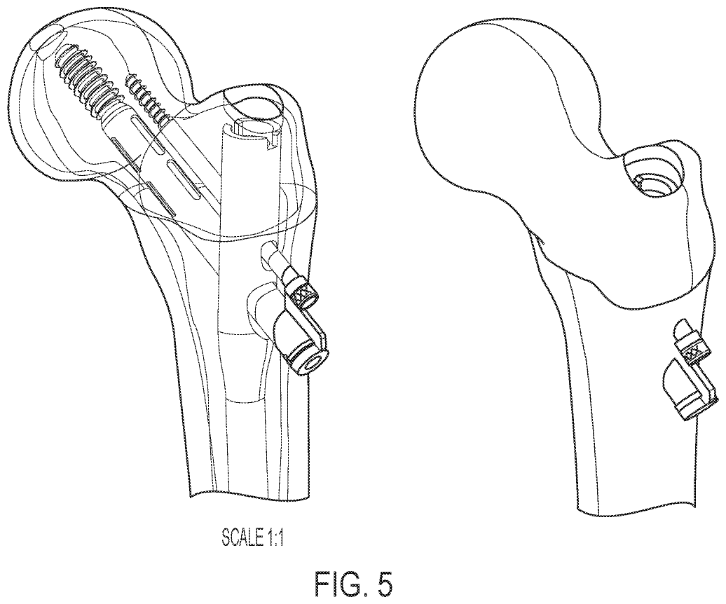

### DEFINITIONS

The instant invention is most clearly understood with reference to the following definitions.

As used herein, the singular form “a,” “an,” and “the” include plural references unless the context clearly dictates otherwise.

As used in the specification and claims, the terms “comprises,” “comprising,” “containing,” “having,” and the like can have the meaning ascribed to them in U.S. patent law and can mean “includes,” “including,” and the like.

As used herein, the term “later” refers to a component that is located relatively away from the midline of a subject's body.

As used herein, the term “medial” refers to a component that is located relatively toward the midline of a subject's body.

Unless specifically stated or obvious from context, the term “or,” as used herein, is understood to be inclusive.

### DETAILED DESCRIPTION OF THE INVENTION

Trochanteric fracture nails are frequently used in the treatment of a proximal femoral fracture. Trochanteric fracture nails, including a lagscrew, are universally designed as dynamic constructs allowing the lag screw, embedded in the femoral head of a patient, to collapse down into the nail as the fracture compresses. In one aspect, the present invention relates to locking in a nail that already has (or is capable of having) a second screw in the proximal nail shaft such as a laterally inserted screw. In some embodiments, the second screw is used as a mounting point for a device (e.g., an off-set clip) that protrudes to cover or make contact with a lateral aspect of the lag-screw in a way that prevents the lag-screw from sliding laterally. The attachment of the off-set clip to the laterally inserted screw prevents or limits the lag-screw from moving without shifting the laterally inserted screw, which may be threaded into the nail for resistance.

In some aspects, the present invention relates to a lag-screw-retaining clip. In another aspect, the present invention relates to a trochanteric fracture nail system comprising a lag-screw-retaining clip which limits the lag screw from moving without shifting the laterally inserted screw. In yet another aspect, the present invention relates to method of treating a proximal femoral fracture in a subject in need thereof.

### Trochanteric Fracture Nail System

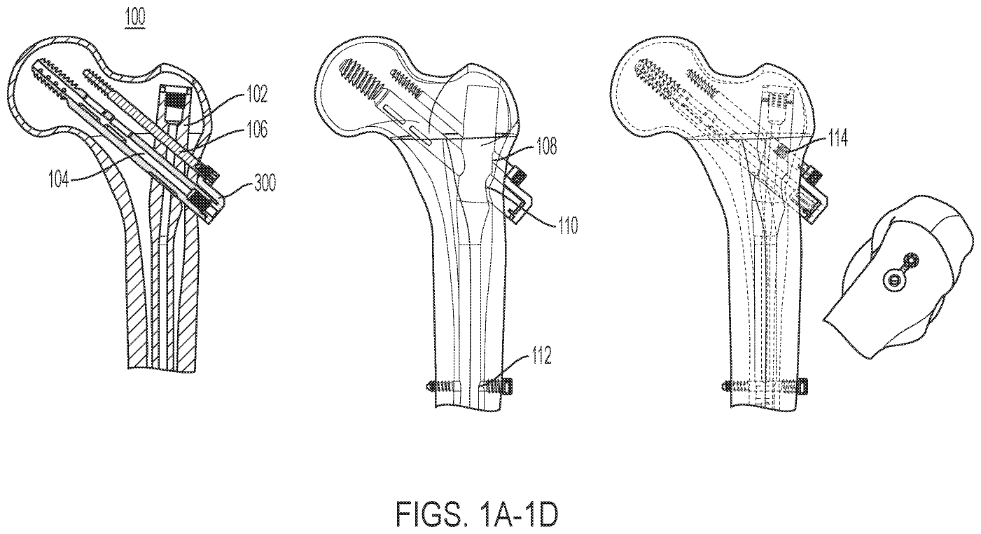

Referring to FIGS. 1A-1D, a trochanteric fracture nail system 100 is provided. Trochanteric fracture nail system 100 includes a trochanteric fracture nail 102, a lag screw 104 adapted and configured for fixation medially through the trochanteric fracture nail 102, a laterally inserted screw 106 adapted and configured for fixation medially through the trochanteric fracture nail, and a lag-screw-retaining clip 300.

Lag screw 104 and laterally inserted screw 106 can be adapted for fixation parallel to each other. In some embodiments, trochanteric fracture nail 102 comprises a first parallel bore 108 and a second parallel bore 110. Lag screw 104 may be inserted through second parallel bore 110 and laterally inserted screw 106 may be inserted through first parallel bore 108 such that trochanteric fracture nail system 100 is configured for placement through the femur of a subject in need thereof. In certain embodiments, trochanteric fracture nail 102 further comprises a third bore 112 distal to first parallel bore 108 and second parallel bore 110 (FIG. 1B). A medical-grade screw may be inserted through the femur of a subject in need thereof, through third bore 112, and through the opposite side of the subject's femur in order to provide additional support to a proximal femoral fracture during healing. In some embodiments, laterally inserted screw 106 comprises one or more threads 114, which can be threaded into trochanteric fracture nail 102.

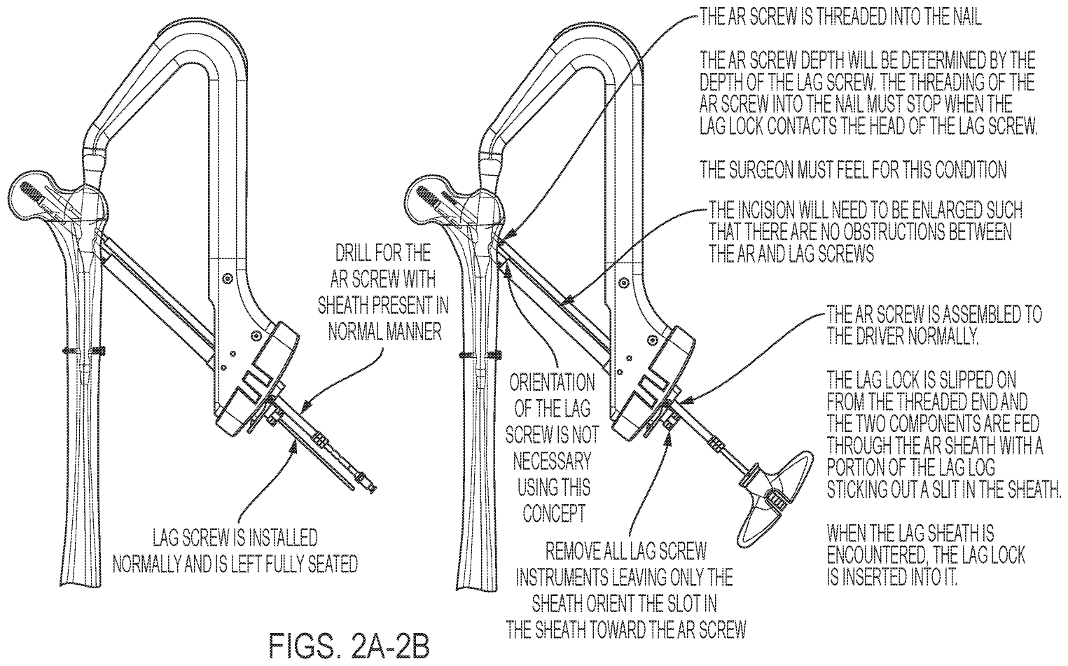

In some embodiments, trochanteric fracture nail system 100 further comprises a guide. FIGS. 2A and 2B depict trochanteric fracture nail system 100 with the guide, wherein the guide comprises parallel bores for placement of lag screw 104 and laterally inserted screw 106 through a subject's femur and through trochanteric fracture nail 102. In some embodiments, a sheath may be inserted into the parallel bores of the guide before placement of lag screw 104 and laterally inserted screw 106. In some embodiments, the sheath may act to hold lag screw 104 and laterally inserted screw 106 in a desired position as the screws are placed through a subject's femur and through trochanteric fracture nail 102. The guide may be adapted and configured to engage with a superior end of the trochanteric fracture nail.

### Lag-Screw-Retaining Clip

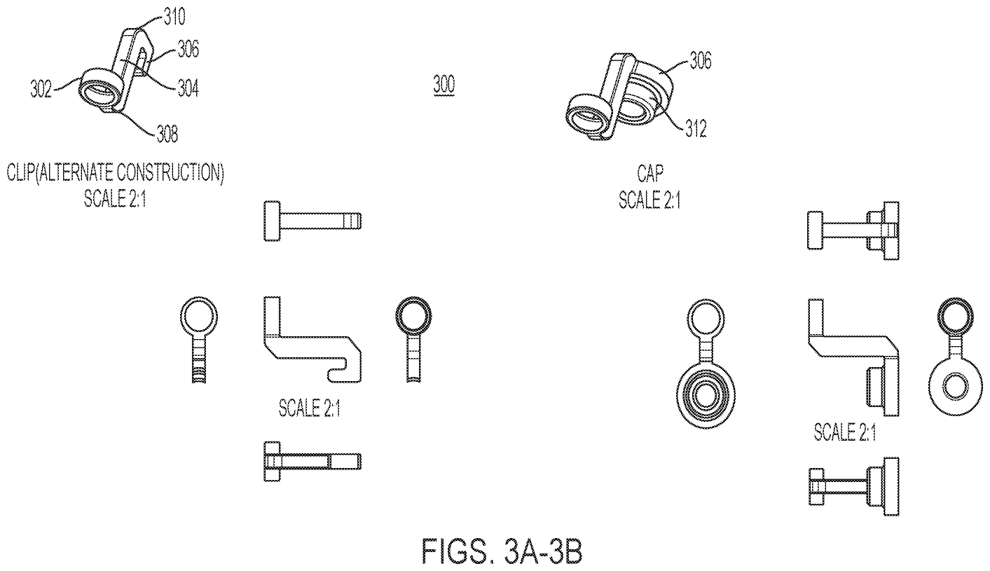

Referring now to FIGS. 3A and 3B, the lag-screw-retaining clip 300 is provided. Clip 300 includes a medial flange 302, an axial offset member 304 coupled to the medial flange 302, the axial offset member comprising a first, medial end coupled to the medial flange and a second, lateral end, and a lateral flange 306 coupled to the second, lateral end of the axial offset member 304.

Medial flange 302 is adapted and configured for engagement medially under a head of laterally inserted screw 106 of trochanteric fracture nail system 100. Lateral flange 306 is adapted and configured to rest against a lateral end of lag screw 104 of trochanteric fracture nail system 100 and resist lateral movement of the lag screw. Lateral flange 306 may further comprise a complementary geometry adapted and configured to engage with the lateral end of lag screw 104.

Medial flange 302 and lateral flange 306 may be positioned at right angles with respect to axial offset member 304. In some embodiments, medial flange 302 is an annular washer. Axial offset member 304 can include a medial bevel 308 and/or a lateral bevel 310 (FIG. 3A). In some embodiments, lateral flange 306 is an annular washer, forming a lag-screw-retaining clip with a cap (FIG. 3B). Lag-screw-retaining clip 300 can further comprise a medial boss 312 adapted and configured to engage with the lateral end of the lag screw (FIG. 3B).

### Materials

The components of trochanteric fracture nail system 100 and lag-screw-retaining clip 300 can be made of any biocompatible material known to a person of skill in the art. In some embodiments, one or more components of nail system 100 or clip 300 are made from a biocompatible metal. Exemplary biocompatible metals include, but are not limited to, stainless steel, titanium, titanium alloy, cobalt-chromium alloy, and combinations thereof In another embodiment, one or more components of nail system 100 or clip 300 are made from a biocompatible plastic. Exemplary biocompatible plastics include, but are not limited to, polyvinyl chloride (PVC), polyethylene, polycarbonate, polyether ether ketone (PEEK), polyetherimide (PEI), polypropylene, polysulfone, polyurethane, and combinations thereof.

### Method of Treating a Proximal Femoral Fracture

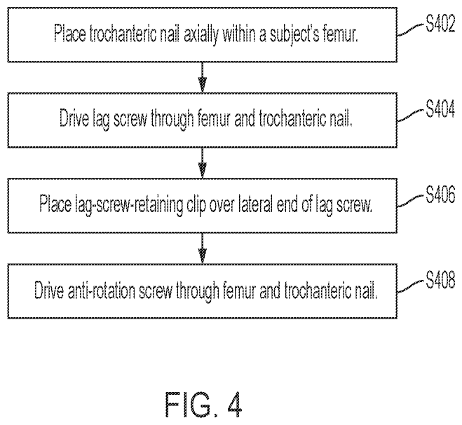

A flowchart depicting an exemplary method of treating a proximal femoral fracture in a subject in need thereof is shown in FIG. 4.

In step S402, a trochanteric fracture nail is placed axially within a subject's femur (e.g., after boring with a surgical drill bit). The trochanteric fracture nail can be nail 102. In some embodiments, a superior end of nail 102 is engaged with a guide wherein the guide comprises parallel bores.

In step S404, a lag screw is driven through the femur and through the trochanteric fracture nail. In some embodiments, lag screw 104 is driven through trochanteric fracture nail 102. In some embodiments, lag screw 104 is driven through second parallel bore 110 of nail 102. In certain embodiments, the lag screw is advanced through a first parallel bore of a guide that is engaged with a superior end of the trochanteric fracture nail. A guidewire and/or imaging (e.g, fluoroscopy) can be utilized to confirm accurate placement of a bore and associated lag screw.

In step S406, a lag-screw-retaining clip comprising an axial offset member comprising a first, medial end coupled to a medial flange and a second, lateral end coupled to a lateral flange is provided and the medial flange is placed laterally over a lateral end of the lag screw such that the medial flange lies medial to the lateral flange. In some embodiments, the lag-screw-retaining clip is clip 300, comprising medial flange 302, axial offset member 304, and lateral flange 306.

The lag-screw-retaining clip can be placed over the shaft of a laterally inserted screw before insertion into the subject such the lag-screw-retaining clip is secured by the head of the laterally inserted screw. In such an embodiment, the lag-screw-retaining clip can rotate about the laterally inserted screw at least until tightening and/or engagement with the lag screw.

In another embodiment, the lag-screw-retaining clip can be positively attached to the lag screw before advancement of the laterally inserted screw. For example, the lag-screw-retaining clip could be threaded to an end of the lag screw. For example, the lag screw could include male or female threads that permit threaded coupling with the lag-screw-retaining clip, either before or after the lag screw is driven.

In step S408, a laterally inserted screw is driven through the femur and through the trochanteric fracture nail such that: the medial flange of the lag-screw-retaining clip is laterally impinged by the laterally inserted screw and the lateral flange of the lag-screw-retaining clip laterally impinges the lag screw. The laterally inserted screw can be screw 106. In some embodiments, laterally inserted screw 106 is driven through trochanteric fracture nail 102. In some embodiments, laterally inserted screw 106 is driven through first parallel bore 108 of nail 102. The laterally inserted screw can be driven through the nail until the lag-screw-retaining clip contacts and impinges the head of the lag screw. In certain embodiments, the laterally inserted screw is advanced through a second parallel bore of a guide that is engaged with a superior end of the trochanteric fracture nail. A guidewire and/or imaging (e.g, fluoroscopy) can be utilized to confirm accurate placement of a bore and associated laterally inserted screw. The lag-screw-retaining clip can be advanced over one or more guidewires.

In some embodiments, the exemplary method further comprises the step of inserting a medical grade screw through the femur of a subject in need thereof, through a third bore in the trochanteric fracture nail, and through the opposite side of the subject's femur. In certain embodiments, the medical grade screw may provide additional support to the proximal femoral fracture during healing.

In embodiments wherein a guide is engaged to the superior end of the trochanteric fracture nail during the exemplary method, at the completion of step S408, the guide can be disengaged from the superior end of the nail after the lag screw and laterally inserted screw are advanced.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A lag-screw-retaining clip comprising:
a medial flange adapted and configured for engagement medially under a head of a laterally inserted screw of a trochanteric fracture nail system;
an axial offset member coupled to the medial flange, the axial offset member comprising:
a first, medial end coupled to the medial flange; and
a second, lateral end; and
a lateral flange coupled to the second, lateral end of the axial offset member, the lateral flange adapted and configured to rest against a lateral end of a lag screw of the trochanteric fracture nail system and resist lateral movement of the lag screw.

2. The lag-screw-retaining clip of claim 1, wherein the medial flange and the lateral flange are positioned at right angles with respect to the axial offset member.

3. The lag-screw-retaining clip of claim 1, wherein the medial flange is an annular washer.

4. The lag-screw-retaining clip of claim 1, wherein the lateral flange is an annular washer.

5. The lag-screw-retaining clip of claim 1, wherein the lateral flange comprises a medial boss adapted and configured to engage with the lateral end of the lag screw.

6. The lag-screw-retaining clip of claim 1, wherein the lateral flange comprises a complementary geometry adapted and configured to engage with the lateral end of the lag screw.

7. The lag-screw-retaining clip of claim 1, wherein the lag-screw-retaining clip consists of a biocompatible material.

8. The lag-screw-retaining clip of claim 7, wherein the biocompatible material is selected from the group consisting of: titanium alloy, stainless steel, and PEEK.

9. The lag-screw-retaining clip of claim 1, wherein the axial offset member comprises a medial bevel.

10. The lag-screw-retaining clip of claim 1, wherein the axial offset member comprises a lateral bevel.

11. A trochanteric fracture nail system comprising:
a trochanteric fracture nail;
a lag screw adapted and configured for fixation medially through the trochanteric fracture nail;
a laterally inserted screw adapted and configured for fixation medially through the trochanteric fracture nail; and
the lag-screw-retaining clip according to claim 1.

12. The trochanteric fracture nail system of claim 11, wherein the lag screw and the laterally inserted screw are adapted for fixation parallel to each other.

13. The trochanteric fracture nail system of claim 11, wherein the trochanteric fracture nail comprises two parallel bores.

14. The trochanteric fracture nail system of claim 11, further comprising:
a guide comprising parallel bores for placement of the lag screw and the laterally inserted screw through a subject's femur and through the trochanteric fracture nail.

15. The trochanteric fracture nail system of claim 14, wherein the guide is adapted and configured to engage with a superior end of the trochanteric fracture nail.

16. A method of treating a proximal femoral fracture in a subject in need of treatment, the method comprising:
placing a trochanteric fracture nail axially within the subject's femur;
driving a lag screw through the femur and through the trochanteric fracture nail;
placing the lateral flange of the lag-screw-retaining clip according to claim 1 laterally over a lateral end of the lag screw such that the medial flange lies medial to the lateral flange; and
driving a laterally inserted screw through the femur and through the trochanteric fracture nail such that:
the medial flange of the lag-screw-retaining clip is laterally impinged by the laterally inserted screw; and
the lateral flange of the lag-screw-retaining clip laterally impinges the lag screw.
